(19) United States
(12) Patent Application Publication
(10) Pub. No.: US 2005/0106740 A1
(43) Pub. Date: May 19, 2005

(54) Methods, systems and devices for performing analytical protocols

(76) Inventors: Barry E. Boyes, Wilmington, DE (US); Nina Zolotarjova, Wilmington, DE (US); Gordon Nicol, Middletown, DE (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/713,761

(22) Filed: Nov. 13, 2003

(51) Int. Cl. G01N 30/02

(52) U.S. Cl. 436/86; 436/161; 422/59

(57) ABSTRACT

Methods, systems and device for performing analytical protocols are provided. Embodiments of the subject methods include sequentially contacting a sample with at least a first stationary phase and a second stationary phase under chromatographic conditions to at least determine the binding identity of the at least one constituent, where the specificity of the first stationary phase for at least one constituent present in the sample is at least uncertain and the specificity of the second stationary phase for the at least one constituent is certain. Also provided are systems and devices that include at least the first and second stationary phases. Kits for use in practicing the subject methods are also provided.

### FIELD OF THE INVENTION

The field of this invention is analytical protocols, specifically chromatography protocols and more specifically liquid chromatography.

### BACKGROUND OF THE INVENTION

Analysis of constituents such as low abundance constituents present in a complex sample mixture often requires the specific removal of other constituents, such as higher abundance constituents, from the mixture, in order to detect and characterize the constituents of interest. For example, in the case of a complex protein sample mixture, one or more high abundance proteins may be present in an amount ranging from about 2% to about 60% of the total protein present. However, these high abundance proteins may confound the detection and characterization of the lower abundance proteins and thus need to be removed from the mixture. However, removal of constituents from a sample is not limited to the above-described removal of high abundance constituents and may be required for a variety of reasons such as the removal of contaminants or other interfering substances from a sample.

Many techniques are conventionally employed in the analysis of sample constituents, e.g., proteins and the like, present in complex mixtures. For example, such techniques include one and two dimensional gel electrophoresis, multidimensional liquid chromatography and mass spectroscopic protocols. However, these conventional protocols suffer from limitations that necessitate the removal of unwanted constituents prior to use. For example, many protocols suffer from dynamic range limitations due to the presence of high abundant constituents, as well as suffering from limitations in their ability to selectively detect lower abundant constituents or their derivatives due to the presence of other constituents. For example, in analyzing complex protein samples, the challenges of characterizing proteins present in plasma or serum will be apparent. In such samples, proteins and peptides of interest are present in amounts ranging from about 55% of the total protein (e.g., serum albumin) to amounts of less than about 10⁻¹⁰ of the total protein and as such it is important in the characterization of these proteins to selectively remove other constituents from the sample mixture.

Various forms of interactive solid phase liquid chromatography (“LC”) are conventionally employed for the removal of constituents, such high abundance proteins, from a sample mixture. However, these conventionally used protocols suffer from inadequate selectivity for removal of particular constituents, e.g., high abundance proteins, and often result in the removal of constituents of interest during the depletion of the unwanted target constituents and/or do not result in the complete removal of the unwanted constituents. Unfortunately, the degree of constituent removal is often not known until after the protocol for the analysis of the constituent of interest is performed, thus wasting time, labor and sample.

As is apparent, a key step in designing these solid phase protocols is the selection of a specific stationary phase and mobile phase combination, hereafter referred to as “a separation system”, that may be used in the removal of constituents in a sample, which maximizes the chances of binding targeted constituents such as high abundance proteins, while minimizing the chances of binding constituents not intended to be bound, while at the same time minimizes the time and expense involved in stationary phase selection and optimization. In practice, designing an optimized stationary phase typically involves iterating the stationary phase and various protocol reagents (mobile phases, elution buffers, etc.) and parameters (temperature, flow rate, etc.) one or more times to find the stationary phase that is best suited for a given protocol. Such iterations are costly and time consuming.

Accordingly, there continues to be an interest in the development of new methods and systems to isolate a constituent of interest from a sample and particularly methods and systems for designing and optimizing materials used in chromatographic protocols for isolating a target constituent of interest. Of particular interest is the development of methods and separation systems that easily and effectively assess the binding properties of chromatographic materials, are easy to use, cost effective, and which minimize the time and expense involved in stationary phase development and optimization.

References of Interest:

References of interest include: U.S. Pat. Nos. 4,722,896; 5,567,317; 5,801,225; 6,495,016; 5,567,317; 4,963,263; 4,722,896 and U.S. patent application Ser. Nos. 2003/0000835; 2002/0127739 and 2002/0127739; as well as international publication no. WO 02/055654.

### SUMMARY OF THE INVENTION

Methods, systems and device for performing analytical protocols are provided. Embodiments of the subject methods include sequentially contacting a sample with at least a first stationary phase and a second stationary phase under chromatographic conditions to at least determine the binding identity of the at least one constituent, where the specificity of the first stationary phase for at least one constituent present in the sample is at least uncertain and the specificity of the second stationary phase for the at least one constituent is certain. Also provided are systems and devices that include at least the first and second stationary phases. Kits for use in practicing the subject methods are also provided.

### BRIEF DESCRIPTIONS OF THE DRAWINGS

FIGS. 1A and 1B show an exemplary embodiment of a system of the subject invention employing one or more columns to retain the subject stationary phases. Specifically, FIG. 1A shows an exemplary embodiment of a system employing two separate columns wherein a first column includes a first stationary phase and a second column includes a second stationary phase and FIG. 1B shows an exemplary embodiment of a system employing a single column wherein at least a first stationary phase and a second column are present in the sample column.

FIG. 2 shows an exemplary embodiment of a system of the subject invention employing a microfluidic device to retain the subject stationary phases.

FIG. 3 shows the results of an SDS-PAGE analysis of protein components in fractions obtained by using the subject methods.

### DEFINITIONS

The term “stationary phase” as used herein refers to an immobile phase or non-fluid phase employed in an analytical protocol such as a chromatography protocol.

The term “mobile phase” as used herein refers to the fluid phase that is employed to move a sample through or over a stationary phase in an analytical protocol such as a chromatography protocol.

The term “separation system” as used herein broadly refers to a particular stationary phase/mobile phase combination used in a separation protocol such as a chromatography protocol.

The term “sample” as used herein relates to a material or mixture of materials, typically, although not necessarily, in fluid form, containing one or more constituents, i.e., components or analytes, of interest.

“Optional” or “optionally” means that the subsequently described circumstance may or may not occur, so that the description includes instances where the circumstance occurs and instances where it does not.

A “computer-based system” refers to the hardware means, software means, and data storage means used to perform certain functions and/or analyze the information of the present invention. The minimum hardware of the computer-based systems of the present invention may include a central processing unit (CPU), input means, output means, and data storage means. A skilled artisan can readily appreciate that any one of the currently available computer-based systems are suitable for use in the present invention. The data storage means may include any manufacture including a recording of information relating to the subject invention, or memory access means that can access such a manufacture.

### DETAILED DESCRIPTION OF THE INVENTION

Methods, systems and device for performing analytical protocols are provided. Embodiments of the subject methods include sequentially contacting a sample with at least a first stationary phase and a second stationary phase under chromatographic conditions to at least determine the binding identity of the at least one constituent, where the specificity of the first stationary phase for at least one constituent present in the sample is at least uncertain and the specificity of the second stationary phase for the at least one constituent is certain. Also provided are systems and devices that include at least the first and second stationary phases. Kits for use in practicing the subject methods are also provided.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

Overview

As noted above, embodiments of the subject methods are characterized by employing at least a first stationary phase and second stationary phase. A feature of certain embodiments of the subject invention is that at least one of the stationary phases is of at least uncertain specificity for at least one of the constituents present in a sample and the specificity of at least one other stationary phase for the at least one constituent is certain or at least more certain than the specificity of the uncertain stationary phase.

As will be described in greater detail below, the order in which the stationary phases are employed, i.e., the order in which a sample is contacted with the stationary phases, in the subject invention may vary. For example, in certain embodiments the sample of interest will be first contacted with a stationary phase having an uncertain specificity for at least one constituent present in the sample and then a resultant binding fraction is contacted with a stationary phase where the specificity for the at least one constituent present in the sample is certain. In certain other embodiments the sample of interest will be first contacted with a stationary phase where the specificity for at least one constituent present in the sample is certain and then a resultant binding fraction is contacted with a stationary phase where the specificity for the at least one constituent present in the sample in uncertain. Accordingly, the subject methods are versatile in this regard and as such may be adapted for use in a variety of different applications as will be described in greater detail below.

In further describing the subject invention, the subject methods are described first, followed by a-review of systems and devices that may be employed in the practice of the subject methods. Finally, kits for use in practicing the subject methods are provided.

Methods

As summarized above, embodiments of the subject methods include sequentially contacting a sample with at least a first and second stationary phase. Accordingly, in the practice of embodiments of the subject methods at least one constituent present in the contacted sample binds to the stationary phases. As noted above, a feature of the subject invention is that the specificity or selectivity (herein used interchangeably) for at least one constituent present in the sample (e.g., a “target” constituent”) is at least uncertain, e.g., unknown, for at least one of the stationary phases and certain or at least more certain for at least one other of the stationary phases. By “uncertain specificity” is meant that some aspect of the binding specificity (e.g., the degree of binding specificity, and the like) of the stationary phase with regards to at least one constituent present in the sample is at least uncertain, e.g., unknown, to the person practicing the subject methods. For example, it may be known that the first stationary phase is capable of binding a given constituent, but the degree of binding (i.e., how well or whether the “intended” or “target” constituent is not being completely bound such that some intended constituents are passing through or over the stationary phase and/or the whether other or rather any “unintended” or “non-target” constituents are being bound) may not be known or there may be some uncertainty as to the degree of binding. By “certain specificity” is meant that the binding specificity of the stationary phase with regards to at least one constituent present in the sample is certain (e.g., known) or at least more certain than the specificity of the unknown stationary phase, to the person practicing the subject methods. In certain embodiments the certainty of the specificity may also include knowledge or certainty that the stationary phase has a higher specificity for at least one constituent present in the sample than the “uncertain” stationary phase, i.e., certain and uncertain, e.g., known and unknown, in such instances may refer to the certainty, e.g., knowing, that one of the stationary phases has a higher specificity for at least one constituent than does another stationary phase.

As the binding specificity of at least one of the stationary phases is uncertain, e.g., unknown, in certain embodiments this stationary phase may be described as a “test” stationary phase, e.g., if an object of the subject methods is to evaluate or characterize the stationary phase in regards to its binding specificity. The subject invention may be employed in variety of applications, as will be further described below. For example, in certain embodiments information related to the binding specificity of at least one of the stationary phases of at least uncertain specificity may be obtained from the practice of the subject methods which may be used in the development or optimization of a future protocol, e.g., to optimize the stationary phase or to determine a suitable stationary phase, for future protocols. Accordingly, embodiments of the methods of the subject invention may be characterized as methods of determining binding identity of a given constituent. By “binding identity” is meant at least one aspect (e.g., a chemical aspect, physical aspect, biological activity aspect, and the like) of at least one binding partner member of a specific binding complex with respect to at least one binding assay. For example, such a binding identity may include the molecular structure of a molecule, e.g., the molecular structure of a protein component in a protein ligand binding interaction.

Embodiments of the subject methods are described primarily with reference to a single first stationary phase and a single second stationary phase. However, such is for convenience only and is in no way intended to limit the subject invention as the subject method may be employed in parallel protocols, e.g., to evaluate a number of different stationary phases. For example, a number of different stationary phases having known or unknown specificity may be employed in parallel fashion or rather at the same time or simultaneously, e.g., by employing multiple chromatography columns in parallel (e.g., using conventional 96, 384, 1024, etc., well arrays of columns), etc. Some or all of the various stationary phases which may be employed in a parallel fashion may be the same or may differ in one or more physical and/or chemical properties, e.g., prepared with various compositions of ligands and/or various binding or elution conditions, etc. It is further contemplated that the subject methods may be performed using microfluidic devices which may facilitate the performance of parallel protocols. Such microfluidic devices are known in the art and are characterized by having at least one fluid channel having at least one dimension (e.g., length, depth, width, etc.,) on the order of nanometers such as described in U.S. Pat. No. 6,495,016, and U.S. patent application Ser. No. 2003/0000835, the disclosures of which are herein incorporated by reference.

In practicing the subject methods, a sample containing, or at least suspected of containing, at least one constituent of interest is contacted first with one of the stationary phases to provide a binding fraction of the sample that includes at least one constituent. The bound constituent(s) may be intentionally bound (i.e., may be targets) or may have been unintentionally bound such that the binding thereof was not intended. The bound constituent may all be the same or may differ in one or more respects. The binding fraction is then separated from the stationary phase and contacted with the other stationary phase in a sequential manner. As noted above, the order of contact of the stationary phases may vary depending on the particular protocol, and as such in general the steps of the subject methods may be characterized as (a) first contacting a stationary phase having specificity that is at least uncertain for at least one constituent present in a sample and then contacting a another or second stationary phase having specificity that is certain or known for the at least one constituent, or (b) first contacting a stationary phase having a specificity that is certain for at least one constituent present in a sample and then contacting another or second stationary phase having specificity that is at least uncertain or unknown for the at least one constituent.

Before further describing the subject methods, the stationary phases of the subject invention are now described in greater detail, followed by a description of representative samples that may be used with the subject stationary phases in the practice of the subject methods.

Exemplary Stationary Phases

As summarized above, the subject invention includes at least two stationary phases of the subject invention such that at least a first stationary phase and a second stationary phase are employed in the practice of the subject invention. The stationary phases may be any suitable stationary phases and will vary depending on the particular protocol being performed. By “stationary phase” is meant an immobile phase. The immobile phase may be contrasted with the mobile phase or eluent, i.e., the liquid or fluid phase described in greater detail below. In general, the first and second stationary phases of the subject invention include a solid support or substrate having a bonded phase, where the bonded phase is attached, associated, connected or otherwise coupled or-linked to the solid support. The bonded phase is selected to bind at least one constituent present in a sample contacted with the stationary phase i.e., the bonding phase may be described as a binding agent such as a binding member of a specific binding complex, e.g., a specific binding pair or interaction.

The stationary phases may be a solid, a bonded or coated phase on a solid support, or a wall-coated phase. In many embodiments, the first and second stationary phases are made up of a plurality of particles, e.g., as is known in the art, for example as employed for liquid chromatography protocols, and may be porous or non-porous. If porous, the average pore size and total porosity of a given stationary phase, i.e., the ratio of the volume of interstices to the volume of the solid particles, is chosen to optimize the particular separation procedure being performed. The porosity of a given stationary phase of the subject invention may vary depending on the particular separation protocol being performed. Embodiments may include porous substrates having an average pore size ranging from about 100 to about 10,000, e.g., from about 300 to about 3,000, e.g., from 300 to about 1,000.

A variety of materials may be employed for the solid supports, substrates or matrices of the stationary phases and need not be the same material for at least a first stationary phase of at least uncertain specificity and at least one other stationary phase where the specificity is certain. Representative materials include, but are not limited to, magnetic particles, silica (e.g., SiO₂), alumina (e.g., Al₂O₃), any other suitable metal oxides including transition metal oxides, as well as polymeric materials and collapsible polymers such as styrene, polyacrylamide, poly(styrene-divinylbenzene (PS-DVB), cellulose, sugars and sugar polymers (e.g., agarose such as Sepharose™ available from Amersham Biosciences, dextran, and the like), silica-coated polymers, membranes, organo modified metal or transition metal oxides (hybrid) and continuous metal oxide or chemically modified metal oxide monolithic structures. These materials are exemplary only and are in no way intended to limit the scope of the invention and other chromatography solid supports, resins or matrices may be employed as well such as other affinity chromatography resins, etc.

As described above, at least the stationary phases of certain and uncertain specificity include a bonded phase that is chosen to bind one or more target constituents present in the sample, i.e., the bonded phases of at least a first and second stationary phase are complementary binding substances that bind their “binding partner constituent(s)” or complementary constituent(s) present in the sample. The bonded phases (also referred to as “ligand”) are stably associated with the solid support of the stationary phases by any suitable techniques where the ligands may be covalently bound, adsorbed or otherwise attached to the solid support. The ligands of the stationary phases usually reversibly bind at least one constituent so that any constituent(s) that bind to the ligands may be eluted therefrom by employing a suitable elution protocol, e.g., using a suitable elution buffer.

The ligands may be general or group ligands or specific ligands. General ligands have affinity for a group of related constituents or substances having at least some structural or functional similarity rather than for a single constituent or substance as is the case with a specific ligand. As such, a general ligand will bind to several substances (e.g., a class or population of ligand binding proteins e.g., enzymes binding to cofactors or their analogs), with similar or identical kinetic or thermodynamic characteristics. In contrast, specific ligands have a higher relative affinity (thermodynamic or kinetic characteristics) for, or will bind specifically to, a specific binding partner as opposed to other potential or related binding partners.

Representative ligands include, but are not limited to, antibodies or binding fragments thereof (e.g., Fv, F(ab)₂, Fab) and include mono-, bi- and poly-functional antibodies, diabodies, minibodies, antigens, dyes, derivatives of AMP NADH and NADPH, Concanavalin A, proteins such as lectins (e.g., lentil lectin, wheat germ lectin, and the like), protein A, protein G, benzamidine, phenylboronic acid, glycoproteins, peptides, nucleic acids (DNA, RNA), nucleosides, nucleotides, methyl esters of various amino acids, D-amino acids, vitamins, receptors, enzymes, soybean trypsin inhibitors, inorganic chemicals, organic chemicals. The ligand density of the stationary phases will vary depending on the particular ligand, solid support, particular protocol, etc.

In certain embodiments the bonded phase may include chlorotriazine affinity ligands also known as dye molecules such as Cibacron Blue F3G-A dye or Reactive Blue 2. Of particular interest in certain embodiments are stationary phases of unknown specificity that include such dye molecules, i.e., the subject invention may be employed to evaluate the binding-specificity of such stationary phases. Also of interest are immunoaffinity ligands (i.e., antibodies or binding fragments thereof (e.g., Fv, F(ab)₂, F(ab) as noted above) and of particular interest in certain embodiments are stationary phases of known specificity that include such immunoaffinity ligands. In certain embodiments at least a first stationary phase includes dye molecule ligands such as Cibacron Blue and a second stationary phase includes immunoaffinity ligands. Such embodiments employing dye molecule ligands and/or immunoaffinity ligands are particularly useful when binding high abundance constituents such as human serum albumin (“HSA”) constituents from a complex protein sample, e.g., to characterize the binding specificity of the first stationary phase with respect to HSA or to remove the albumin from the sample to concentrate the sample with respect to other constituents.

In certain embodiments, a stationary phase of at least certain specificity for at least one constituent present in the sample has a high degree of specificity, or rather a higher relative affinity, for that constituent than for other constituents of the sample. By “high degree of specificity” is meant that the relative binding affinity of this stationary phase for at least one constituent of a sample is higher than the binding affinity for at least one other constituent of the sample, e.g., an unwanted, or contaminant, constituent of the sample. High affinity binding may be in the range of about 10⁸ to 10¹⁰ L/mol or more, whereas low affinity may be in the range of range of about 10³ L/mol or less (e.g., see U.S. Pat. No. 4,963,263, incorporated herein by reference). Thus, a given stationary phase may be considered to be highly specific for at least one constituent present in a sample if the relative affinity for that constituent is significantly greater than the affinity of other, e.g., unwanted constituents present in the sample. In this aspect, a significantly greater relative affinity may be on the order of a 10-fold to 10⁶-fold difference, or greater in certain embodiments. Such differences in relative affinity, or specificity, are strongly dependent on environmental conditions, for example, the pH or ionic composition of the solution in which association of the targeted constituent and the stationary phase occurs. Moreover, specificity is also a function of the relative concentration levels and nature of constituents present in the sample, such that competition for binding may occur between low affinity binding constituents that may be present at high concentration, relative to high affinity constituents that may be present at low concentration. One particularly effective way to achieve a highly specific binding is to employ immunochromatography ligands in which specific antibodies for a targeted constituent may be employed. Accordingly, as noted above, in many embodiments at least one of the stationary phases of the subject invention is an immunoaffinity stationary phase such that the ligands employed are antibody or binding fractions thereof.

In many embodiments, at least one of the stationary phases (e.g., the stationary phase of at least certain specificity) will have a greater specificity for at least one constituent present in a sample than does at least one other stationary phase (e.g., the stationary phase having uncertain specificity). For example, stationary phase embodiments may include a specificity-certain stationary phase that has a high degree of specificity for at least one constituent present in a sample and which has a greater specificity for the at least one constituent present in a sample than does at least one other stationary phase employed in the binding identity determination protocol.

In coupling the ligands to the substrate to provide a suitable stationary phase, any suitable coupling technique may be employed to attach a ligand to a given solid support or substrate, e.g., cyano bromide activation and the like, where such protocols are known in the art. In certain embodiments a particular ligand employed in a given stationary phase may be attached to the solid support or matrix via a linker or spacer arm, e.g., if the active site of-the ligand is located deep within the molecule. Accordingly, in certain embodiments a suitable “linker” or “spacer arm” may be interposed between the matrix and ligand to facilitate effective binding.

Typically, the second stationary phases are present in a suitable apparatus for performing the subject invention. For example, each may be in a different chromatography column or both may be in the same chromatography column or in certain embodiments each stationary phase may be present in a channel (the same or different) of a microfluidic device.

Exemplary Samples and Constituents

As reviewed above, one or more constituents are present in a sample, where the term “sample” as used herein refers to a material or mixture of materials, typically, although not necessarily, in fluid form, containing one or more constituents of interest. A sample may be any suitable sample that includes a target constituent such as naturally occurring and synthetically derived samples related to plants, animals and the like, where the sample and/or the constituent-may-be pre-processed prior to contact with the first stationary phase, e.g., may be amplified, denatured, filtered, centrifuged, fractionated, etc. For example, representative, exemplary samples may include, but are not limited to, biological fluids such as whole blood, serum, plasma, organ fluids, semen, bile, perspiration, cell suspensions, cell lysates, cytoplasm, protein solutions, urine, tears, cerebrospinal fluid, and the like, as well as non-biological, industrial and environmental fluids such as water (e.g., waste water and water from a municipal water source treated and untreated), buffer, organic fluids, and the like.

The samples include, or are suspected of including, at least one constituent or analyte (herein used interchangeably), where the constituent(s) may be naturally present in the sample, e.g., in the sample's natural state, or may be added to a sample, e.g., by a researcher, or the like. The particular constituents present in a given sample will depend on a variety of factors such as the type of sample, etc., The constituents thus include any suitable constituent(s) including, but not limited to, proteins (e.g., blood and serum proteins such as albumin, nucleases, polymerases, interferons, and the like), peptides, polypeptides, glycoproteins, saccharides (mono- poly- and oligo- saccharides) nucleic acids (DNA and RNA-single and double stranded), lipids, phospholipids, glycolipids, fullerene compounds, carboxylic acids, vitamins, immunoglobins, catecholamines, purines, pyrimidines, nucleotides, various pharmaceuticals such as polar pharmaceuticals, microorganisms, bacteria, viruses, etc. In certain embodiments the constituents include high abundance proteins (e.g., serum albumin), e.g., present in a serum sample. It is to be understood that the above-described representative samples and constituents are exemplary only and are in no way intended to limit the scope of the invention. Prior to being contacted with a stationary phase of the subject invention some or all of the constituent(s) present in the sample may be processed prior to such combining.

The number of different constituents present in a sample contacted with the first stationary phase may vary depending on a variety of factors, e.g., the nature of the sample, etc. Accordingly, the number of target constituents, i.e., the number of different constituents that are intended to be bound to the first stationary phase (while the remaining constituents are not bound, or pass by), may be as few as about one type of constituent or more, e.g., may be as many as ten types or more, e.g., hundreds of types or more e.g., thousands of types or more, e.g., tens of thousands of types or more, e.g., hundreds of thousands of types or more, millions of types or more.

For example, it may be desired to evaluate the binding specificity of a stationary phase for a single “target” constituent, e.g., serum albumin or it may be desired to evaluate the specificity of a stationary phase for a group or a class of constituents, (e.g., the group or class of target constituents share a common characteristic, e.g., members of a particular class of proteins) such that more than one type of constituent, i.e., different constituents, is targeted. In certain embodiments, it may be desired to evaluate the binding specificity of a stationary phase for high abundance proteins present in a serum sample.

Usually, a mobile phase or fluid (also known as a “binding buffer” or “loading buffer” in certain instances) is combined with the constituent-containing sample prior to being contacted with the stationary phases of the subject invention to move the constituent-containing sample over or through the particular stationary phase. The mobile phase may vary depending on the particular protocol being performed and may be made of one or more different mobile phase fluids. A suitable mobile phase or “binding buffer” promotes the specific binding of the target constituent to the ligands of the stationary phase. In many embodiments, the mobile phase is of neutral to weak alkaline pH and is of sufficient ionic strength to minimize any non-specific ionic interactions. One such mobile phase is a low salt phosphate buffer, e.g., PBS, having a pH between about 7.2 to about 7.4. In certain embodiments, e.g., using a Cibacron Blue (“CB”) stationary phase, a suitable loading buffer may be 50 mM K₂PO₄, with or without the addition of neutral salts, for example NaCl or KCl in the range of about 50 to about 500 mM, pH 6-8. Other representative fluids that may make-up a given mobile phase include, but are not limited to, water (i.e., 100% water), and aqueous-based fluids, various phosphate buffers, neutral salt solutions, methanol, heptafluorobutyric acid (HFBA), acetonitrile, formic acid, N,N′-diethylamine (DEA), tetrahydrofuran, trifluoroacetic acid (TFA), acetone, dichloromethane, tetrahydrofuran, hexane, n-heptane, propanol, ethanol, isopropanol, and the like. Various other components or additives may be included with the fluids where such components include, but are not limited to, surfactants, a suitable buffering system, and the like.

Once a sample having at least one constituent is combined with a suitable mobile phase or binding buffer, the constituent-containing sample/mobile phase mixture is contacted with one of the stationary phases of the subject invention under suitable conditions, e.g., suitable chromatographic conditions, to bind a fraction of the sample that includes at least one of the constituents present in the sample, i.e., to isolate the at least one constituent(s) from other constituents present in the sample. In this manner, one or more constituents are bound or otherwise retained for a period of time by this stationary phase while other constituents are passed through or over the stationary phase and eluted, e.g., for further analysis. As these other constituents do not bind to the stationary phase and instead “flow through” the stationary phase (as well as any non-specifically bound constituents), they may be described as the “flow through” constituents. The term “contact” is used herein in the broadest sense to mean any type of combining action which brings the constituents into sufficiently close contact with the stationary phase and more particularly with the ligands of the stationary phase such that a binding interaction will occur between the ligand and any binding partner present in the sample. The constituent-containing sample/mobile phase mixture is flowed over or through the stationary phase under conditions suitable to bind at least one constituent present in the binding fraction binds to bind to its binding partner of the stationary phase.

After all of the sample has been contacted to the stationary phase, the column is typically washed one or more times to remove any non-specifically bound constituents from the stationary phase, but not any bound constituents. Such washing may be accomplished with one or more suitable volumes of mobile phase or binding buffer or a suitable buffer without sample until baseline is established. Wash volumes may range from about 1 to about 20 column volumes or more, e.g., about 2 to about 15 column volumes.

The one or more bound constituents (i.e., the binding fraction), whether intentionally bound or unintentionally bound, are then separated from the stationary phase by being eluted from the stationary phase. In certain embodiments, the flow through constituents are also collected and analyzed using a suitable protocol, e.g., to determine if any constituents that were intended to bind to the stationary phase failed to bind and instead passed over or through the stationary phase. Any suitable technique may be employed to elute the binding fraction such as disrupting the binding by changing pH, ionic strength, dielectric constant or temperature using a suitable elution buffer or mobile phase. In certain embodiments, this may be accomplished by employing a suitable mobile phase or “elution buffer” designed to reduce the interaction between bound constituent and the stationary phase, e.g., by exploiting the polarity of a mobile phase (using a less polar or more polar mobile phase than previously used). One suitable elution buffer, e.g., for eluting constituent from a Cibacron Blue stationary phase, is a phosphate buffer such as 50 mM K₂PO₄/1.5 M KCl, pH 7. In certain embodiments elution may be accomplished by varying the proportions of the fluids of the mobile phase previously employed (e.g., a gradient elution) or by employing a different mobile phase all together. For example, this elution may be accomplished by employing a mobile phase gradient that increases in concentration of an organic modifier (e.g., acetonitrile or methanol) relative to water to elute the constituents, e.g., in order of increasing hydrophobicity.

Once the binding fraction is separated from the stationary phase it is contacted with at least one other stationary phase, such that if the previously contacted stationary phase was a stationary phase having at least uncertain specificity, e.g., unknown specificity, then the binding fraction is then contacted with a stationary phase having certain, e.g., known, specificity and conversely if the previously contacted stationary phase was a stationary phase having a certain specificity then the binding fraction is then contacted-with a stationary phase having an at least uncertain specificity. In certain embodiments, these first and second stationary phases may be in fluid communication (e.g., in the same device or connected by a fluid channel or conduit) thereby enabling the continuous flow of a substance, e.g., mobile phase, elution buffer, binding fraction, and the like, between the two stationary phases without interruption, e.g., continuously Such may be accomplished, for example, by employing a single chromatography column packed with both stationary phases, e.g., in contact with each other (e.g., sequentially in contact with each other) or by employing a suitable microfluidic device wherein two or more microfluidic channel are fluidly connected or the two stationary phases are present in the same microfluidic channel. In such continuous flow through embodiments, prior to subject the product of the first column to the second column the first column product may be adjusted or altered in one or more respects.

Typically, the binding fraction is combined with a mobile phase, fluid or binding buffer as analogous to that described above and flowed over or through the stationary phase under conditions suitable to bind at least one constituent present in the binding fraction. In this manner, at least one constituent present in the binding fraction binds to the stationary phase and any unbound constituents flow through or over the stationary phase and are collected.

Accordingly, following this second binding step any unbound constituents or rather any “flow through” constituents are collected, where such usually includes washing the stationary phase one or more times after all of the binding fraction has been applied thereto to remove any non-specifically bound constituents. Such washing may be accomplished with one or more suitable volumes of mobile phase or a suitable buffer without sample until baseline is established. Wash volumes may range from about 1 to about 20 column volumes or more, e.g., about 2 to about 15 column volumes.

The flow through constituents may then be analyzed and identified using any suitable analysis protocol such as by detecting a change in UV-VIS absorption at a set wavelength, by refractive index, by fluorescence after excitation with a suitable wavelength, by electrochemical response, by one- or two-dimensional gel electrophoresis (e.g., SDS-PAGE), by matrix assisted laser desorption/ionization mass spectroscopy, by liquid chromatography/mass spectroscopy, biomolecular interaction (e.g., surface plasmon resonance etc.), immunochemical analysis (e.g., immunoassay, western blotting), various additional spectroscopic approaches (e.g., nuclear magnetic resonance (“NMR”), circular dichroism (“CD”), etc.), and the like, for further analysis of the constituents where such analysis may provide information related to a particular stationary phase or to the sample itself.

Following the wash protocols of this second binding step, the bound constituent may be separated from this second stationary phase and may be detected and identified using any suitable detection protocol such as by detecting a change in UV-VIS absorption at a set wavelength, by refractive index, by fluorescence after excitation with a suitable wavelength, by electrochemical response, by one- or two-dimensional gel electrophoresis (e.g., SDS-PAGE), by matrix assisted laser desorption/ionization mass spectroscopy, by liquid chromatography/mass spectroscopy, biomolecular interaction (e.g., surface plasmon resonance etc.), immunochemical analysis (e.g., immunoassay, western blotting), various additional spectroscopic approaches (e.g., nuclear magnetic resonance (“NMR”), circular dichroism (“CD”), etc.), and the like. Any suitable elution fluid may be employed to remove the bound constituents from the second stationary phase such as described above.

Accordingly, in certain embodiments the results of an analysis of the flow through and/or bound constituents may be related to the binding specificity of one of the stationary phases. For example, the presence of the other constituents that did not bind to the second stationary phase (i.e., the flow through constituents) may indicate the degree of specificity of the stationary phase of unknown specificity, e.g., may indicate that this stationary phase was not highly specific for a given or “target” constituent and consequently this stationary phase bound constituents other than the target as evidenced by their presence in the flow through fraction.

The subject methods may provide information that may be used in a decision regarding whether to use a particular stationary phase in combination with a particular mobile phase in future protocols (e.g., whether a stationary phase/mobile phase combination is sufficiently specific for a given constituent) or whether a stationary phase/mobile phase combination requires further optimization prior to being used in a future protocol or whether the particular stationary phase/mobile phase combination is not useable in a future protocol at all.

In certain embodiments, it may be desirable to remove certain constituents (e.g., interfering constituents) from a sample (targets) and not others, e.g., to concentrate the remaining constituents for further analysis where such removal of constituents may be necessary to achieve a suitable analysis of these remaining constituents such as a suitable separation using a gel separation technique or the like. However, if the stationary phase is binding constituents that are intended to flow through the column and not be bound and used in a future protocol such as gel electrophoresis, any future analysis of the flow through constituents may be adversely affected by this unintentional binding. Accordingly, the subject methods may be employed to evaluate the binding properties of a particular stationary phase/mobile phase combination in regards to selective removal of certain constituents from a sample.

Certain embodiments may include obtaining data related to the above-described methods, for example data related to the binding specificity of a given separation system, data related to constituents identified by the above-described methods, etc., and further processing or manipulating the data and/or forwarding, e.g., by communication, or transmitting the data. Such results may be raw results or may be processed results such as obtained by comparing a result or data point to a predetermined reference or standard and forming conclusions based on this comparison such as whether or not a particular constituent is present in the sample, whether or not a particular constituent is bound by one or both of the stationary phases, etc. The results (processed or not) may be forwarded (such as by communication) to a remote location if desired, and received there for further use (such as further processing), as now described in greater detail.

In certain embodiments, data relating to the subject methods, may be transmitted to a remote location. By “remote location” it is meant a location other than the location at which the separation protocol and/or constituent detection occurs. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient electronic or telecommunications means may be employed for transmitting the data, e.g., facsimile, modem, Internet, etc.

Utility

The subject methods may be employed in a variety of applications that include, but are not limited to, determining the binding identity of at least one constituent in a sample. Embodiments include evaluating the selectivity of one of the stationary phases for at least one constituent present in the sample, evaluating the selectivity of a pharmaceutical agent for a given constituent (e.g., protein) present in a sample, separating a sub-class of proteins from a class of proteins, evaluating one or more chromatography parameters such as temperature, pH, elution buffers, binding buffers, etc. Each of these representative applications is now described below.

Stationary Phase Binding Selectivity Evaluation

As noted above, the subject methods may be employed in the evaluation of the selectivity of a stationary phase, i.e., a “test stationary phase” for at least one constituent present in a sample. In accordance with the subject methods, this may be accomplished by first contacting the sample with a-first or test stationary phase having unknown specificity for at least one constituent present in the sample and then contacting the binding fraction separated from this first stationary phase with a second stationary phase having a known specificity for the at least one constituent. Such may be employed to determine the constituents (e.g., type, amount, etc.) that bind to the test stationary phase, e.g., whether it binds constituents other than a specific or rather “target” (i.e., intended) constituent and if so, to determine the identity of these other “non-target” bound constituents. Such may also be employed in the determination of the degree of binding of the target or whether any targets do not bind to the test stationary phase and instead flow through.

The target constituent(s) may be any suitable target constituent as described above, e.g., high abundant-proteins in a serum sample (e.g., serum albumin or HSA), a specific protein intended to be bound by a pharmaceutical agent, or any other constituent such as contaminants or interfering substances present in a sample. For example, in certain embodiments the subject invention may be employed to assess the binding specificity of a pharmaceutical agent, e.g., a pharmaceutical agent under development, for a particular protein. For example, a pharmaceutical agent may be intended to bind or be highly selective for a particular protein (i.e., an intended or target protein), but not others. Accordingly, the subject methods may be employed as a means to efficiently and effectively determine the binding identity, e.g., specificity, of a target protein for a particular pharmaceutical agent as well as, to determine if the pharmaceutical agent binds other proteins as well.

Accordingly, the particular first stationary phase employed will vary depending on the particulars of the protocol such as the target constituent, sample, etc., however as describe above the binding specificity of this first stationary phase is unknown. For example, where the target is high abundant proteins such as HSA present in a serum sample and the protocol includes an evaluation of the specificity of the first stationary phase to bind H-SA (and/or to enrich low abundant proteins in a sample), the first stationary phase may include dye molecule ligands such as Cibacron Blue ligands or the like. In other embodiments such as to evaluate the binding specificity of a pharmaceutical agent for a particular target protein (i.e., the target), the pharmaceutical agent under evaluation may be employed as the ligands of the first stationary phase such that in such embodiments the pharmaceutical agent of interest is immobilized (without detriment to its binding activity) on a suitable substrate to provide a first stationary phase.

A sample containing at least the target of interest is contacted with a first stationary phase having at least uncertain, e.g., unknown, specificity for at least one constituent or target in the sample under chromatographic conditions to bind a fraction of the sample that includes the at least one constituent to the first stationary phase, where the specificity of the first stationary phase for at least one target is at least uncertain. As noted above, contacting may be accomplished in any convenient manner such as injection, pipette, etc. As described above, any suitable chromatographic conditions may be employed.

After the sample has been contacted with the first stationary phase to provide a binding fraction of the sample that includes at least one constituent, (which may or may not be the target constituent), the first stationary phase is usually washed as described above to remove any non-specifically bound constituents. In certain embodiments, the constituents that did not bind to the first stationary phase, and instead passed through the first stationary phase unimpeded, i.e., the flow through constituents, may be collected and analyzed and identified.

For example, such analysis and identification of flow through constituents may provide information about the binding specificity of the first stationary phase. For example, identification of target constituents in the flow through provides an indication of the degree of specificity of the first stationary phase such that the first stationary phase did not bind all (or possibly any) of the target constituents as they are present in the flow through.

Regardless of whether the flow through is analyzed or not, the subject methods further include separating the binding fraction from the first stationary phase, where such may be accomplished in any suitable manner such as described above. This binding fraction is then contacted with the second stationary phase where the binding specificity for at least the target constituent is certain, e.g., known. The second stationary phase typically has a high specificity for the target(s), e.g., in many embodiments the second stationary phase is an immunochromatographic stationary phase that has a high specificity for the target constituents. The particulars of the second stationary phase will vary depending on a variety of factors. For example, where the target is HSA, the second stationary phase is chosen to selectively bind HSA, e.g., may include anti-HSA antibody (or binding fractions). In those embodiments where the first stationary phase included a pharmaceutical agent, the second stationary phase is chosen to selectively bind the intended target protein of the pharmaceutical agent such as an antibody or binding fraction thereof having selectivity for the target protein.

Accordingly, the binding fraction is contacted with the second stationary phase under chromatographic conditions where contacting may be accomplished in any convenient manner such as injection, pipette, etc. As described above, any suitable chromatographic conditions may be employed.

The flow through constituents are collected and analyzed to provide information about the binding specificity of the first stationary phase or rather the stationary phase that had unknown specificity. Any suitable analysis protocol may be employed as described above (e.g., by detecting a change in UV-VIS absorption at a set wavelength, by refractive index, by fluorescence after excitation with a suitable wavelength, by electrochemical response, by one- or two-dimensional gel electrophoresis (e.g., SDS-PAGE), by matrix assisted laser desorption/ionization mass spectroscopy, by liquid chromatography/mass spectroscopy, biomolecular interaction (e.g., surface plasmon resonance etc.), immunochemical analysis (e.g., immunoassay, western blotting), various additional spectroscopic approaches (e.g., nuclear magnetic resonance (“NMR”), circular dichroism (“CD”), etc.). For example, any constituents present in the flow through indicates the constituents were bound by the first stationary phase, i.e., the first stationary phase bound constituents other than the target constituent(s).

For example, in an evaluation of the binding specificity of the first stationary phase to bind HSA present in a serum sample, constituents detected in the flow through from the second stationary phase indicates the first stationary phase bound constituents other than HSA. In an evaluation of the binding specificity of a pharmaceutical agent for a target protein, proteins detected in the flow through from the second stationary phase indicates the first stationary phase bound proteins other than the target protein.

Pharmaceutical Agent Binding Selectivity Evaluation

As noted above, the subject methods may be employed in the evaluation of the selectivity of a pharmaceutical agent for at least one constituent present in a sample. While the above-described method that included first contacting a sample with a stationary phase of at least uncertain binding specificity and then contacting the binding fraction therefrom with another stationary phase having certain or known specificity may be employed in an evaluation of the selectivity of a pharmaceutical agent as reviewed above, an alternative embodiment is now described. In general, this alternative embodiment includes first contacting a sample with a stationary phase where the binding specificity for at least one constituent present in a sample is certain or known and then contacting the binding fraction therefrom with another stationary phase having uncertain or unknown specificity for the at least one constituent.

Accordingly, a sample containing at least one target constituent is contacted with the a first stationary phase of certain or known binding specificity for at least the target constituent present in a sample under chromatographic conditions to bind a fraction of the sample that includes at least the target to this stationary phase. In this particular embodiment, this stationary phase may have a high specificity for the target(s), e.g., an immunoaffinity stationary phase that has a high specificity for the target constituent(s), and thus may serve to isolate the target from other constituents present in the sample. The particulars of this stationary phase will vary depending on a variety of factors such as the particular target constituent, etc. The first contacted stationary phase in this embodiment may be chosen to selectively bind the intended target protein of the pharmaceutical agent such that in many instances the ligands of this stationary phase are antibodies or binding fractions thereof having a high specificity for the target protein.

Contacting may be accomplished in any convenient manner as described above such as injection, pipette, etc. As described above, any suitable chromatographic conditions may be employed.

After the sample has been contacted with this stationary phase to provide a binding fraction of the sample that includes at least the target constituent, the stationary phase is usually washed as described above to remove any non-specifically bound constituents.

Following the wash steps, the subject methods include separating the binding fraction from the stationary phase, where such may be accomplished in any suitable manner such as described above. This binding fraction is then contacted with another or second stationary phase or test stationary phase (i.e., having uncertain or unknown specificity for the target). The particulars of the second stationary phase will vary depending on a-variety of factors, but this-stationary phase includes the pharmaceutical agent under evaluation.

Accordingly, the binding fraction is contacted with the pharmaceutical agent of the stationary phase under chromatographic conditions where contacting may be accomplished in any convenient manner such as injection, pipette, etc. As described above, any suitable chromatographic conditions may be employed.

The flow through from the second stationary phase constituents are collected and analyzed to provide information about the binding specificity of the pharmaceutical agent. Any suitable analysis protocol may be employed as described above (by detecting a change in UV-VIS absorption at a set wavelength, by refractive index, by fluorescence after excitation with a suitable wavelength, by electrochemical response, by one- or two-dimensional gel electrophoresis (e.g., SDS-PAGE), by matrix assisted laser desorption/ionization mass spectroscopy, by liquid chromatography/mass spectroscopy, biomolecular interaction (e.g., surface plasmon resonance etc.), immunochemical analysis (e.g., immunoassay, western blotting), various additional spectroscopic approaches (e.g., nuclear magnetic resonance (“NMR”), circular dichroism (“CD”), etc.). For example, target protein present in the flow through of the second stationary phase indicates that the pharmaceutical agent did not bind all, if any, of the target protein. The subject methods may be employed, for example, for pharmaceutical drug screening and drug development purposes.

Embodiments also include methods of determining the specificity of a pharmaceutical agent for at least one constituent present in a sample where the methods include sequentially contacting a sample with at least a first stationary phase and a second stationary phase under chromatographic conditions such that the specificity of the first stationary phase for at least one constituent present in the sample is at least uncertain, and the specificity of the second stationary phase for the at least one constituent is certain. Such embodiments may include first contacting a sample with a stationary phase of certain specificity for at least one constituent present in the sample, and then contacting the sample with a second stationary phase of at least uncertain specificity for the at least one constituent, where in certain embodiments this stationary phase of at least uncertain specificity includes a pharmaceutical agent of interest (i.e., a pharmaceutical agent whose specificity is at least unknown for at least one constituent present in the sample). In certain embodiments, such embodiments include contacting the sample with the stationary phase of certain specificity to bind a fraction of the sample that comprises at least one constituent to that stationary phase, separating the un-bound fraction from the stationary phase of certain specificity (i.e., the flow-through or rather any constituents that did not bind to the stationary phase of certain specificity), and then contacting the un-bound fraction with the stationary phase of at least uncertain specificity. Any constituents that did not bind to the second-contacted stationary phase (i.e., the stationary phase of at least uncertain specificity) may then be analyzed using any suitable analysis protocol such as those described above to determine the specificity of the pharmaceutical agent for at least one sample constituent.

Separating a Sub-Class of Proteins From a Class of Proteins

The subject methods may be employed to separate a sub-class of constituents, e.g., a sub class of proteins, from a class or population of constituents, e.g., a class of proteins, present in a sample. More specifically, the subject methods find use in the separation of a class of similar or like constituents such as any series of functionally related proteins that share ligand-binding properties, but not immunochemically-specific binding sites of the target constituent.

For example, a first stationary phase may be employed that binds one or more functionally related proteins such as all proteins in a sample that share ligand-binding properties, e.g., all proteins that have a particular carbohydrate moiety that binds to lectin ligands. Accordingly, to remove or separate a sub-class or target from the first binding fraction, a sample containing the class of proteins of interest is contacted with this first stationary phase having ligands that at least bind the class of proteins. As noted above, a first stationary phase having lectin ligands may be employed to bind all proteins with a particular carbohydrate moiety.

Contacting may be accomplished in any convenient manner as described above such as injection, pipette, etc. As described above, any suitable chromatographic conditions may be employed.

After the sample has been contacted with the first stationary phase to provide a binding fraction of the sample that includes at least the target protein class of interest, the first stationary phase is usually washed as described above to remove any non-specifically bound constituents.

Following the wash steps, the binding fraction is separated from the first stationary phase, where such may be accomplished in any suitable manner such as described above. This binding fraction is then contacted with the second stationary phase. The second stationary phase is chosen to selectively bind one or more sub-classes of the class and not bind any of the other members of the class. In this manner, one or more sub-classes may be isolated from the class. In other words, the binding fraction that includes the class of proteins is contacted with a second stationary phase that is more specific to the sub-class or target than the first stationary phase and is not as specific to the other members of the class, e.g., the second stationary phase may include antibody or antibody fraction ligands specific for the sub-class or target but not the other members of the class.

In many embodiments, the second stationary phase is of certain or known specificity for the target(s) of interest (and the first phase may be of uncertain specificity) such an immunoaffinity stationary phase, i.e., includes antibodies or binding fractions thereof, having a high specificity for the one or more sub-classes intended to be separated.

Accordingly, the binding fraction that contains the class of proteins is contacted with the second stationary phase under chromatographic conditions where contacting may be accomplished in any convenient manner such as injection, pipette, etc. As described above, any suitable chromatographic conditions may be employed.

The bound sub-classes and/or the flow through from the second stationary phase may then be collected and analyzed, e.g., to provide information about the binding specificity of the first stationary phase for the class and/or sub-class, etc. It will be apparent that the above-described subject methods may be employed to separate sub-classes and/or other members of the class to be used in future protocols.

Evaluating Chromatography Parameters

The subject methods may be employed to evaluate various chromatography parameters and their effect on the binding specificity of a given separation system, e.g., to optimize parameters thereof. Such parameters may include, but are not limited to, binding buffers, eluting buffers, temperature, flow rate, mobile phase composition, etc. Conveniently, a plurality of protocols according to the subject methods may be employed in parallel or simultaneously to minimize the time required to evaluate a given parameter. For example, a given parameter may vary amongst the stationary phases while all other parameters are held constant.

For example, the subject methods may be employed to evaluate a parameter such as a binding buffer that provides the best binding specificity for a target while minimizing non-specific binding. Accordingly, a plurality of first or test stationary phases may be employed, where each is the same (i.e., the only variable is the binding buffer used). The first stationary phase may then be contacted with a sample including at least the target constituent to provide a binding fraction of the sample that includes at least the target constituent in a manner analogous to that described above. Flow through may be collected and analyzed to determine the amount of target that did not bind for each buffer, i.e., to evaluate which buffer promoted the greatest amount of target binding.

The binding fraction may then be separated from the first stationary phase and contacted with a second stationary phase having a high specificity for the target, e.g., a higher specificity than the first stationary phase, in a manner analogous to that described above. In this manner, target present in the binding fraction will bind to the second stationary phase while non-targets will flow through or over the second stationary phase. These constituents that did not bind to the second stationary phase may be collected and analyzed as described above to provide information about the binding specificities of each of the buffers employed, as all other variables remained the same throughout the protocol. For example, a buffer corresponding to a flow through fraction from the second column having the least amount of non-target constituents may indicate an optimum buffer with respect to the other variables as it indicates the first stationary phase/buffer combination bound the least amount of non-targets. As noted above, an analysis of the-flow through from the first stationary phase may be used to indicate which stationary phase/buffer combination bound the most or least amount of target.

Systems

Also provided are systems that may be used in the practice of the subject methods. The systems of the subject invention include a sample having at least one constituent as described above and at least a first stationary phase having an unknown binding specificity for at least once constituent present in the sample and a second-stationary phase having a known specificity for the at least one constituent, as described above. In certain embodiments at least one of the stationary phases includes chlorotriazine affinity ligands, e.g., a CB affinity resin, and/or an immunoaffinity phase. In certain embodiments at least one stationary phase includes a pharmaceutical agent under evaluation, as reviewed above. The stationary phases of the subject invention may be present in a suitable apparatus for performing the subject methods, where representative apparatuses are described in greater detail below.

The subject systems may also include one or more mobile phases or fluids for use in practicing the subject methods such as a binding buffer, elution buffer, wash buffer, and the like. Representative fluids that may be employed in the subject invention are described above, e.g., phosphate buffers and the like.. A given system may include one, two, three or more fluids which may be used alone or in combination with one or more of the other fluids, e.g., employed in combination to provide a given mobile phase. For example, embodiments may include at least a first fluid and a second fluid such that the mobile phase employed may include varying proportions of each fluid such as about 100% of the first fluid and about 0% of the second fluid, followed by lesser percentages of the first fluid and corresponding greater percentages of the second fluid such that the mobile phase may transition to one having about 0% of the first fluid and about 100% of the second fluid. Third, fourth, fifth, etc., fluids may also be incorporated into a protocol in a manner analogous to that described above using two fluids.

Depending on the particular protocol being performed, the pH of the individual fluids, as well as the pH of the mix of any two or more fluids (i.e., the mobile phase) will vary and may be acidic, neutral or basic such that the pH may range from 1-14. As a variety of different fluids may be employed with the subject systems depending on the particular analytical protocol being performed, the fluids may be polar, non-polar, organic, inorganic, etc. Various other components or additives may be included with one or more fluids or may be added to the mobile phase subsequent to the mixing of two or more fluids in a particular proportion where such components include, but are not limited to, surfactants, a suitable buffering system, neutral salts, chaotropic additives, and the like.

Other components of the subject system may include, but are not limited to, fraction collectors, constituent detectors, constituent analyzing components such as electrophoretic gel apparatuses (e.g., a one- or two-dimensional gel electrophoresis system), mass spectroscopy apparatuses, liquid chromatography apparatuses, immunochemical analysis apparatuses, western blotting apparatuses, NMR apparatuses, circular dichroism apparatuses, and the like.

Devices

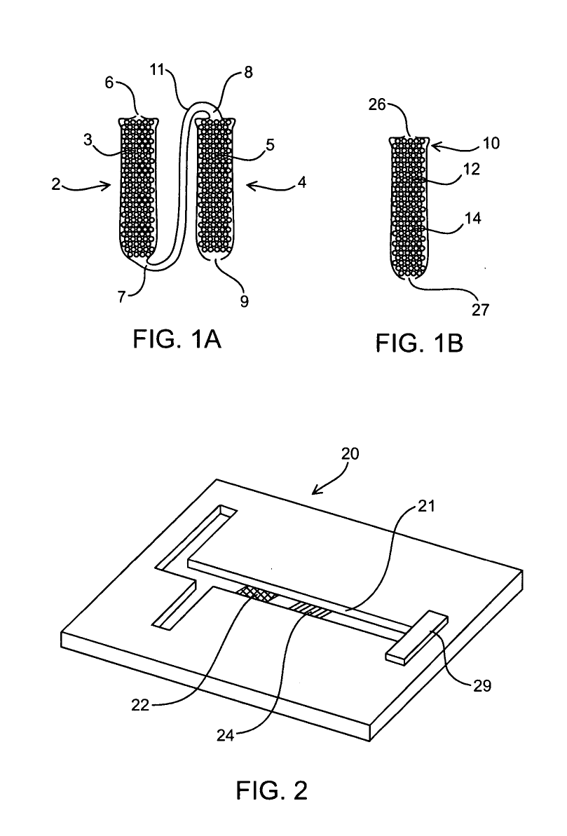

Also provided are devices that may be used in the practice of the subject methods. In general, the devices of the subject invention include at least a first stationary phase having an unknown binding specificity for at least once constituent present in the sample and a second stationary phase having a known specificity for the at least one constituent, as described above. In certain embodiments at least one of the stationary phases includes chlorotriazine affinity ligands,.e.g., a CB affinity resin, and/or an immunoaffinity phase. In certain embodiments at least one stationary phase includes a pharmaceutical agent under evaluation, as reviewed above. 100971 Any suitable apparatus may-be employed, where apparatuses suitable for use in chromatography protocols are of interest. For example, the stationary phases of the subject invention may be present or packed in one or more suitable chromatography columns or associated with a suitable microfluidic device. Where the stationary phases are present in one or more chromatography columns, as shown in FIGS. 1A and 1B, in certain embodiments two separate columns may be employed (FIG. 1A) wherein a first column 2 may be employed having a first stationary phase 3 and a second column may be employed 4 having a second stationary phase 5. In certain embodiments shown in FIG. 1B, a single column 10 may be employed having at least a first stationary phase 12 and a second stationary phase 14 present therein. The columns of the present invention include a fluid inlet port 6, 8 for columns 2 and 4 respectively of FIG. 1A, and fluid inlet port 26 for column 10 of FIG. 1A, for the introduction or ingress of substances to the column. Also provided is a fluid outlet port 7, 9 for columns 2 and 4 respectively of FIG. 1A, and 27 for column 10 of FIG. 1B, for the egress of substances from the column. In certain embodiments employing two separate columns such as shown in FIG. 1A, the columns may be, but not always, fluidly connected such that a fluid communication conduit 11 may exit between the two columns for a continuous or uninterrupted fluid flow between the columns. It will be apparent that the two or more columns employed in the subject binding identity protocols may not be fluidly connected.

Alternative embodiments include microfluidic devices having the subject stationary phases. An exemplary embodiment of such a microfluidic device is shown in FIG. 2 wherein microfluidic device 20 includes at least one flow channel 21 having at least a first stationary phase 22 and a second stationary phase 24 present therein. In this particular embodiment, the stationary phases are shown in the same channel, but in other embodiments the two stationary phases may be present in two different, but fluidly connected channels of a microfluidic device. As noted above, microfluidic devices are known in the art and are characterized by having at least one fluid channel having at least one dimension (e.g., length, depth, width, etc.,) on the order of nanometers such as described in U.S. Pat. No. 6,495,016, and U.S. patent application Ser. No. 2003/0000835, the disclosures of which are herein incorporated by reference. The microfluidic devices may also include a suitable detector 29 positioned directly on the microfluidic device as shown here or may be a separate component.

Kits

Also provided by the subject invention are kits that include at least a first and second stationary phase according to the subject invention and instructions for using the stationary phases in the subject methods. The instructions for using the stationary phases in the subject methods may be printed on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic, magnetic or optical storage data file present on a suitable computer readable storage medium, e.g., CD-ROM, diskette, etc.

In yet other embodiments, the instructions for using the subject stationary phases in the subject methods may not themselves be present in the kit, but means for obtaining the instructions from a remote source, e.g., via the Internet, are provided. An example of this embodiment is a kit that includes a World Wide Web address where the instructions may-be viewed and/or from which the instructions may be downloaded. Some form of access security or identification protocol may be used to limit access to those entitled to use the subject invention.

In addition to the stationary phases, the kits may further include one or more additional components for performing the subject methods, such as suitable mobile phase fluids, e.g., binding buffers, elution buffers, constituent staining reagents, devices for concentrating fractions obtained by the subject methods such as filters, microcentrifugation devices and the like.

### Experimental

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Analysis of Protein Components in Fractions Generated By Employing the Subject Methods

125 μl of human sera was diluted 4-fold with Cibacron Blue (“CB”) resin loading buffer (50 mM K₂PO₄, pH 7) and passed through a first stationary phase made-up of CB resin i.e., CB resin (HiTrap Blue HP, Amersham Pharmacia Biotech AB) at a flow rate of 1 ml/min. The column was washed with 10 column volumes of the loading buffer and bound proteins were eluted with 50 mM K₂PO_{4/}1.5 mM KCl, pH 7. Proteins in the bound fraction were concentrated using Millipore Corp. spin filters (Ultrafree-4, Biomax-5K). Following this procedure, protein content of the fractions was assessed using a BCA protein assay from Pierce Biotechnology and the samples were analyzed by non-reducing SDS-PAGE on 4-20% gradient gel. The protein bands in the gel were visualized by staining with colloidal Commassie Blue (Gel Code Blue, Pierce Biotechnology).

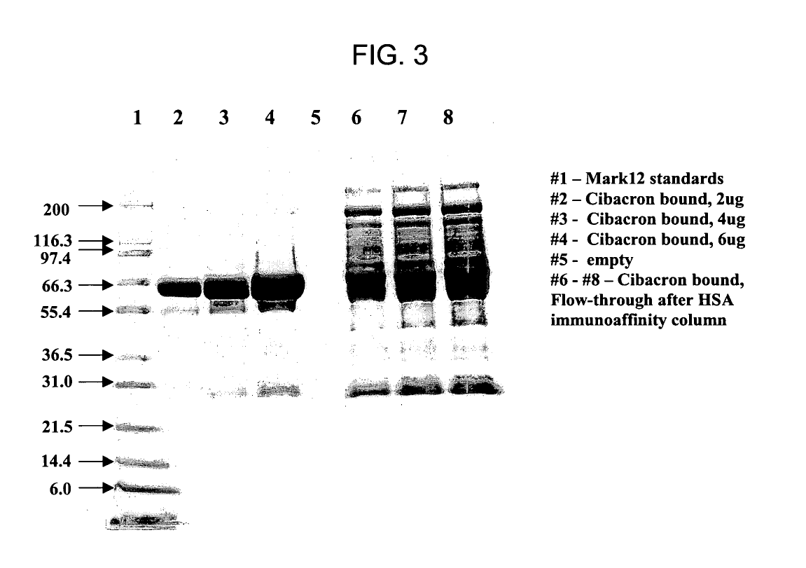

FIG. 3 shows the results of this experiment and lanes 2-4 show the result of this phase of the experiment (lane 1 is a molecular weight standard). As shown in lanes 2-4, serum albumin is clearly the major protein component of the bound fraction, although other minor bands are observed at higher and lower molecular weights. Loading large quantities of the fraction helps visualize these minor bands, however the high protein load required to detect these minor bands (lane 4, 6 μg) compromises the resolution of the electrophoretic separation.

The albumin enriched CB-binding fraction was diluted 4-fold in a neutral phosphate salt buffer solution and then passed over a second anti-HSA affinity stationary phase at a flow rate of 0.25 ml/min. The flow through was concentrated using Millipore Corp. spin filters (Ultrafree-4, Biomax-5K) and this flow through protein mixture was loaded on lanes 6-8 of FIG. 1. As shown, a large number of distinct protein bands are visualized on the gel, ranging widely in apparent molecular weight from 6 kDa to greater than 200 kDa. No band corresponding to the serum albumin is observed in these lanes, although bands with mass above and below are observed. The bands in lanes 6-8 were excised and subjected to tryptic digestion with subsequent mass spectroscopy (“MS”) and LC/MS/MS analysis which identified these CB binding proteins and confirmed their identity to be distinct from serum albumin. Analysis of protein present in the 65 kdal mass range did detect a small amount of HSA, but at a level that is consistent with effective removal of HSA by the immunoaffinity second stationary phase. These results provide binding information about the first stationary phase and specifically that the first stationary phase bound proteins other than HSA.

Accordingly, this experiment shows that the subject invention may be employed to effectively and efficiently fractionate a sample and analyze protein components in the fractions generated using the sequential chromatography protocols of the subject invention to provide binding information about a stationary phase, in this specific example, Cibacron Blue stationary phase.

It is evident from the above results and discussion that the above described invention provides effective methods and devices for characterizing binding identities of targets. The subject invention provides for a number of advantages including, but not limited to, the ability to easily and effectively assess the binding specificity of materials used in chromatographic protocols. Other advantages include ease of use and cost effectiveness. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method comprising sequentially contacting a sample with at least a first stationary phase and a second stationary phase under chromatographic conditions, wherein the specificity of said first stationary phase for at least one constituent present in said sample is at least uncertain, and the specificity of said second stationary phase for said at least one constituent is certain, to at least determine the binding identity of said at least one constituent.

2. The method of claim 1, wherein said method is a method of evaluating the specificity of said first stationary phase for said at least one constituent present in said sample.

3. The method of claim 2, wherein said sample is contacted with said first stationary phase and then said second stationary phase.

4. The method of claim 3, wherein said method further comprises:
(a) contacting said sample with said first stationary phase to bind a fraction of said sample that comprises said at least one constituent;
(b) separating said binding fraction from said first stationary phase; and
(c) contacting said binding fraction with said second stationary phase.

5. The method of claim 3, wherein said first stationary phase comprises a pharmaceutical agent and said method is a method of determining the specificity of said pharmaceutical agent for said at least one constituent present in said sample.

6. The method of claim 1, further comprising analyzing any constituents that did not bind to said second stationary phase.

7. The method of claim 6, wherein said analyzing comprises using at least one of: one dimensional gel electrophoresis, two dimensional gel electrophoresis, matrix assisted laser desorption/ionization mass spectroscopy, liquid chromatography/mass spectroscopy, biomolecular interaction, immunochemical analysis, nuclear magnetic resonance and circular dichroism.

8. The method of claim 1, wherein said method is a method of determining the specificity of a pharmaceutical agent for at least one constituent present in said sample.

9. The method of claim 8, wherein said sample is contacted with said second stationary phase first and said first stationary phase second.

10. The method of claim 9, wherein said method further comprises:
(a) contacting said sample with said second stationary phase to bind a fraction of said sample that comprises at least one constituent;
(b) separating said binding fraction from said second stationary phase; and
(c) contacting said binding fraction with said first stationary phase.

11. The method of claim 9, wherein said first stationary phase comprises said pharmaceutical agent.

12. The method of claim 10, further comprising analyzing any constituents that did not bind to said first stationary phase.

13. The method of claim 12, wherein said analyzing comprises performing at least one of: one dimensional gel electrophoresis, two dimensional gel electrophoresis, matrix assisted laser desorption/ionization mass spectroscopy, liquid chromatography/mass spectroscopy, biomolecular interaction, immunochemical analysis, nuclear magnetic resonance and circular dichroism.

14. The method of claim 1, wherein said sample comprises a population of proteins and said method is a method of separating a sub-population of proteins from said population of proteins.

15. The method of claim 14, wherein said separating comprises:
(a) contacting said sample comprising a class of proteins with said first stationary phase to bind a fraction of said sample that comprises said population of proteins;
(b) collecting said binding fraction from said first stationary phase; and
(c) contacting said binding fraction with said second stationary phase so that said sub-population of proteins binds to said second stationary phase and any remaining members of said population do not bind to said second stationary phase.

16. The method of claim 1, wherein said first and second stationary phases comprise ligands chosen from: antibodies or binding fragments thereof, diabodies, minibodies, antigens, dyes, single chain variants, proteins, glycoproteins, peptides, nucleic acids, vitamins, inorganic chemicals and organic chemicals.

17. The method of claim 16, wherein at least one stationary phase comprises chlorotriazine affinity ligands.

18. The method of claim 16, wherein at least one stationary phase comprises immunoaffinity ligands.

19. The method of claim 16, wherein said first stationary phases comprises chlorotriazine affinity ligands and said second stationary phase comprises immunoaffinity ligands.

20. The method of claim 1, wherein said first and second stationary phases are connected by a conduit.

21. A method of determining the specificity of a pharmaceutical agent for at least one constituent present in said sample comprising sequentially contacting a sample with at least a first stationary phase and a second stationary phase under chromatographic conditions, wherein the specificity of said first stationary phase for at least one constituent present in said sample is at least uncertain, and the specificity of said second stationary phase for said at least one constituent is certain, to at least determine the binding identity of said at least one constituent.

22. The method of claim 21, wherein said sample is contacted with said second stationary phase first and said first stationary phase second.

23. The method of claim 22, wherein said method further comprises:
(a) contacting said sample with said second stationary phase to bind a fraction of said sample that comprises at least one constituent;
(b) separating said non-binding fraction from said second stationary phase; and
(c) contacting said non-binding fraction with said first stationary phase.

24. The method of claim 22, wherein said first stationary phase comprises said pharmaceutical agent.

25. The method of claim 23, further comprising analyzing any constituents that did not bind to said first stationary phase.

26. The method of claim 25, wherein said analyzing comprises performing at least one of: one dimensional gel electrophoresis, two dimensional gel electrophoresis, matrix assisted laser desorption/ionization mass spectroscopy, liquid chromatography/mass spectroscopy, biomolecular interaction, immunochemical analysis, nuclear magnetic resonance and circular dichroism.

27. A method of evaluating the specificity of a stationary phase comprising contacting a sample with at least a first stationary phase and second stationary phase under chromatographic conditions, wherein the specificity of said first stationary phase for at least one constituent present in the sample is at least uncertain, and the specificity of said second stationary phase for said at least one constituent is certain.

28. A method comprising forwarding data representing a result of an analysis step obtained by at least one of the method of claim 6, the method of claim 12 and the method of claim 25.

29. The method according to claim 28, wherein said data is transmitted to a remote location.

30. A method comprising receiving data representing a result of an analysis step obtained by the method of claim 28.

31. A system comprising:
(a) a sample comprising at least one constituent;
(b) a first stationary phase wherein the specificity for at least one constituent present in said sample in at least uncertain; and
(c) a second stationary phase wherein the specificity for said at least one constituent in certain.

32. The system of claim 31, wherein at least one of said stationary phases comprises chlorotriazine affinity ligands.

33. A device comprising a first stationary phase of uncertain specificity for at least one constituent of a sample and a second stationary phase of certain specificity for said at least one constituent of a sample.

34. The device of claim 33, wherein said device is a chromatography column.

35. The device of claim 33, wherein said device is a microfluidic device.

36. A kit comprising:
(a) a first stationary phase of at least uncertain specificity;
(b) a second stationary phase of certain specificity; and
(c) instructions for using the first and second stationary-phases in the method of claim 1.
